(19) United States
(12) Patent Application Publication
(10) Pub. No.: US 2023/0192555 A1
(43) Pub. Date: Jun. 22, 2023

(54) NITROGEN-SULFUR CO-DOPED TI3C2-MXENE NANOSHEET AND PREPARATION METHOD AND APPLICATION THEREOF

(71) Applicant: Civil Aviation University of China, Tianjin (CN)

(72) Inventors: Da Chen, Tianjin (CN); Shuaibin Shao, Tianjin (CN); Meiling Lian, Tianjin (CN)

(73) Assignee: Civil Aviation University of China, Tianjin (CN)

(21) Appl. No.: 17/964,053

(22) Filed: Oct. 12, 2022

(30) Foreign Application Priority Data
Oct. 26, 2021 (CN) 202111244546.8

(51) Int. Cl. C04B 35/56; C04B 35/626; C12Q 1/62; C12Q 1/28

(52) U.S. Cl. CPC C04B 35/5611; C04B 35/62655; C04B 35/6264; C04B 35/6262; C12Q 1/62; C12Q 1/28; C04B 2235/3843

(57) ABSTRACT

The present invention discloses a nitrogen-sulfur co-doped Ti₃C₂-MXene nanosheet and a preparation method and application thereof. Ti₃C₂-MXene is obtained by etching ternary layered carbides of MAX phase through hydrofluoric acid; and then, the nitrogen-sulfur co-doped Ti₃C₂-MXene nanosheet is synthesized by a simple one-step method by taking thiourea as a heteroatom source. The nitrogen-sulfur co-doped Ti₃C₂-MXene nanosheet has a unique two-dimensional layered structure, large specific surface area and abundant heteroatomic catalytic activity sites so that the material presents excellent peroxidase-like activity. The method of the present invention can successfully dope two elements of nitrogen and sulfur in one step on Ti₃C₂-MXene, and can effectively overcome the tedious problem of a step-by-step doping step and the secondary pollution problem of different doping sources to endow peroxidase-like activity for Ti₃C₂-MXene.

### TECHNICAL FIELD

The present invention belongs to the technical field of nano biosensing, relates to a nitrogen-sulfur co-doped Ti₃C₂-MXene nanosheet and a preparation method thereof, and specifically discloses a method for detecting uric acid through simulation of peroxidase activity based on a nitrogen-sulfur co-doped Ti₃C₂-MXene nanosheet.

### BACKGROUND

Uric acid is a main final compound of a purine nucleotide by-product in human metabolism. The uric acid in body fluid is an important biomarker of many diseases. When the uric acid concentration is greater than 0.42 mM in male serum or greater than 0.36 mM in female serum, hyperuricemia is caused. However, hyperuricemia is closely related to gout, arthritis, kidney diseases and cardiovascular diseases. Conventional uric acid detection methods have high cost, long cycle and cumbersome operation, and cannot satisfy the needs of convenient and rapid daily detection. Convenient, rapid, intuitive and effective detection of uric acid based on a calorimetric sensing mechanism through the use of nanomaterial (nanoenzyme) with enzyme-like activity has attracted great attention. Various kinds of nanoenzymes, including precious metal particles, metal oxides, metal sulfides and carbon nanomaterial, have been developed for the construction of colorimetric sensing. However, some inherent disadvantages (low stability, high cost and preparation difficulty) prevent their wide application to a great extent. Therefore, the search for effective enzyme simulation agents becomes an increasingly important target in biological assessment.

Novel two-dimensional transition metal carbide (nitride) (MXene) has good electronic properties, electrochemical properties, optical properties and mechanical properties, and thus is widely applied to the fields of energy storage, nanomedicine and biosensing. Compared with traditional precious metal material, MXene is more stable in physicochemical properties, can be stored for a long time, and is easy to synthesize on a large scale and low in cost. In particular, MXene is also regarded as an effective probe in sensing application, and has the advantages of rapidness, portability and label-free detection. Although remarkable results are achieved by MXene in biosensing, there are still, some challenges to be addressed in the future. Firstly, a preparation method of MXene is relatively simple, which leads to relatively single nature of the obtained MXene and unsatisfactory biosensing performance. To further improve MXene-based sensing performance, several strategies are proposed by researchers, including surface functionalization, acquisition of monolayer nanosheets and formation of composites. However, aggregation and pore deformation may occur in the nanosheets, leading to the loss of active sites in the process of sensing application. Although most precious metals and nanocomposites have high catalytic activity, the characteristics of high cost, poor stability and complex synthesis limit the application outside laboratories.

Therefore, to expand the application potential, it is imperative to synthesize and explore MXene-based nanomaterials with excellent sensing performance. The research progress of MXene in colorimetric sensing will be promoted by regulating the composition and structure through heteroatom doping and changing the electronic performance of carbon materials to enhance the catalytic performance of the materials. It is known that there is currently no published report in literature about the use of non-metal doped MXene as a mimic enzyme for uric acid detection.

### SUMMARY

In view of this, the purpose of the present invention is to provide a nitrogen-sulfur co-doped Ti₃C₂-MXene nanosheet and a preparation method thereof, and further discloses a method for detecting uric acid through simulation of peroxidase activity based on a nitrogen-sulfur co-doped Ti₃C₂-MXene nanosheet; with respect to the problems in the prior art.

It should be noted that on the one hand, the present invention develops an efficient nitrogen-sulfur co-doped Ti₃C₂-MXene mimic enzyme by taking thiourea as a source of heteroatom doping, which can accelerate hydrogen peroxide decomposition to produce —OH; and on the other hand, the content of blood uric acid is detected conveniently, quickly and accurately by observing color change of TMB through the oxidation reduction effect of —OH and uric acid on a chromogenic substrate TMB.

To achieve the above purpose, the present invention adopts the following technical solution:

A nitrogen-sulfur co-doped Ti₃C₂-MXene nanosheet is provided; the nitrogen-sulfur co-doped Ti₃C₂-MXene nanosheet is prepared by a one-step method with thiourea as a source of heteroatom doping; the nitrogen-sulfur co-doped Ti₃C₂-MXene nanosheet has peroxidase-like activity; and the nitrogen-sulfur co-doped Ti₃C₂-MXene nanosheet has an organ-shaped layered structure with a thickness of 6-10 μm, and doped elements are evenly distributed on the nanosheet.

The nitrogen-sulfur co-doped Ti₃C₂-MXene nanosheet has a unique two-dimensional layered structure, large specific surface area and abundant heteroatomic catalytic activity sites so that the material presents excellent peroxidase-like activity. When hydrogen peroxide exists, the material can promote the decomposition to produce hydroxyl radicals (—OH). These —OH can oxidize the chromogenic substrate TMB (colorless) to oxTMB (blue), and then use uric acid to specifically reduce oxTMB (blue) to TMB (colorless) to achieve the quantitative detection of the uric acid.

The present invention characterizes the nitrogen-sulfur co-doped Ti₃C₂-MXene nanosheet by SEM scanning and XPS spectrum.

Another purpose of the present invention is to provide a preparation method of the nitrogen-sulfur co-doped Ti₃C₂-MXene nanosheet.

To achieve the above purpose, the present invention adopts the following technical solution:

The preparation method of the nitrogen-sulfur co-doped Ti₃C₂-MXene nanosheet specifically comprises the steps:

1) slowly adding MAX phase ceramic powder to hydrofluoric acid, and stirring by magnetic force at room temperature to react; after the reaction. washing and centrifuging a corrosion product; washing with absolute ethanol for 3-8 times; and filially, drying the product in a vacuum oven to obtain a Ti₃C₂-MXene nanosheet;

2) grinding and evenly mixing the Ti₃C₂-MXene nanosheet obtained in step 1) and thiourea; then roasting the mixture in an Ar gas atmosphere furnace, and then cooling in the furnace to room temperature; grinding the product again, and centrifugally washing the product with deionized water; and finally drying the product to obtain the nitrogen-sulfur co-doped Ti₃C₂-MXene nanosheet.

Preferably, in the step 1), the reaction mass ratio of the MAX phase ceramic powder and the hydrofluoric acid is 1:4 to 1:8, stirring reaction time is 8-24 h, and a stirring rate is 500-1000 r/min.

Further preferably, drying temperature in the vacuum oven is 40° C.-80° C., and drying time is 8-16 h.

Preferably, in step 2), the mixing mass ratio of the Ti₃C₂-MXene nanosheet and the thiourea is (¼-½): 1, roasting temperature is 300° C.-700° C., and temperature retention time is 4-8 h.

By adopting the above technical solution, the present invention has the following beneficial effects:

The method of the present invention not only can successfully dope two elements of nitrogen and sulfur in one step on Ti₃C₂-MXene, but also can effectively overcome the tedious problem of a step-by-step doping step and the secondary pollution problem of different doping sources, thereby endowing peroxidase-like activity for Ti₃C₂-MXene.

Specifically, the peroxidase-like activity of the nitrogen-sulfur co-doped Ti₃C₂-MXene nanosheet:

50-200 μL of TMB solution (10-25 mM), 50-200 μL of hydrogen peroxide solution (35-50 mM) and 50-200 μL, of nitrogen-sulfur co-doped Ti₃C₂-MXene nanosheet (0.1-0.5 mg/mL) are successively added into 1500-2000 μL of disodium hydrogen phosphate-citrate buffer; a reaction system is evenly mixed; after reaction for 5-20 min, the LTV-VIS absorption spectrum of the mixed solution is determined with, an ultraviolet-visible spectrophotometer; and an absorbance value at a wavelength of 652 nm is recorded.

Another purpose of the present invention is to provide an application of the nitrogen-sulfur co-doped Ti₃C₂-MXene nanosheet in a method for detecting uric acid through simulation of peroxidase activity.

Further, the nitrogen-sulfur co-doped Ti₃C₂-MXene nanosheet has peroxidase-like activity, and the method for detecting uric acid is a colorimetric detection method.

Further, the colorimetric detection method for uric acid comprises the following steps:

50-200 μL, of nitrogen-sulfur co-doped Ti₃C₂-MXene nanosheet (0.1-0.5 mg/mL), 50-200 μL of uric acid solutions with different concentrations, 50 μL of hydrogen peroxide solution (35-50 mM) and 50-200 μL, of TMB (10-25 mM) are successively added into 1500-2000 μL of disodium hydrogen phosphate-citrate buffer: a reaction system is evenly mixed and incubated in a water bath of 30-50° C., for 5-20 min; the UV-VIS absorption spectrum of the mixed solution is determined with an ultraviolet-visible spectrophotometer; and the absorbance at a wavelength of 652 nm is recorded.

Preferably, the disodium hydrogen phosphate-citrate buffer has pH of 4.2 and concentration of 0.14M.

Preferably, after mixing, the incubation temperature of the reaction system is 40 V, and reaction time is 10 min.

In addition, the method for detecting uric acid through simulation of peroxidase activity based on the nitrogen-sulfur co-doped Ti₃C₂-MXene nanosheet not only solves the problems of high cost, long cycle and high operation difficulty of traditional uric acid detection methods and has the advantages of convenience, rapidness, accuracy, high efficiency and low cost, but also achieves accurate quantitative detection in actual samples, wherein the detection range of uric acid is 5-400 μM, which can enrich the quantitative detection of uric acid content in serum samples, and realizes wide application prospects.

It can be known from the above technical solution that compared with the prior art, the present invention provides a nitrogen-sulfur co-doped Ti₃C₂-MXene nanosheet and a preparation method and application thereof, and has the following excellent effects:

1. The present invention prepares the nitrogen-sulfur co-doped Ti₃C₂-MXene by a one-step method by taking thiourea as a source of heteroatom doping, which effectively overcomes the tedious problem of the step-by-step doping step and the secondary pollution problem of different doping sources.

2. In the present invention, the surface inertia of Ti₃C₂-MXene is changed by heteroatom doping so that the nitrogen-sulfur co-doped Ti₃C₂-MXene has excellent peroxidase-like activity.

3. The calorimetric detection method constructed based on the nitrogen-sulfur co-doped Ti₃C₂-MXene nanosheet in the present invention can achieve convenient, rapid, accurate and efficient visualized low-cost uric acid detection, and has high sensitivity to uric acid detection and a detection range of 5-400 μM.

4. A nano biosensing platform disclosed by the present invention can realize quantitative detection of uric acid content in serum samples.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 shows an SEM diagram of a nitrogen-sulfur co-doped Ti₃C₂-MXene nanosheet in embodiment 1 of the present invention;

FIG. 2 shows XPS spectrograms of a nitrogen-sulfur co-doped Ti₃C₂-MXene nanosheet in embodiment 1 of the present invention, wherein (a) XPS full spectrum; (b) C is fine spectrum; (c) N Is fine spectrum; (d) S 2p fine spectrum;

FIG. 3 shows UV-VIS absorption spectra of different reaction systems in embodiment 1 of the present invention;

FIG. 4 shows the steady-state kinetic determination of a nitrogen-sulfur co-doped Ti₃C₂-MXene nanosheet with TMB as substrate in embodiment 1 of the present invention;

FIG. 5 shows the steady-state kinetic determination of a nitrogen-sulfur co-doped Ti₃C₂-MXene nanosheet with H₂O₂ as substrate in embodiment 1 of the present invention;

FIG. 6 shows (a) uric acid concentration-absorbance UV spectrum and (b) uric acid concentration-absorbance standard curve diagram in embodiment 2 of the present invention; and

FIG. 7 shows a selective experimental diagram of a sensing platform for uric acid in embodiment 2 of the present invention.

### DETAILED DESCRIPTION

The technical solution in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a method for detecting uric acid through simulation of peroxidase activity based on a nitrogen-sulfur co-doped Ti₃C₂-MXene nanosheet. The method not only can successfully dope two elements of nitrogen and sulfur in one step on Ti₃C₂-MXene and can effectively overcome the tedious problem of a step-by-step doping step and the secondary pollution problem of different doping sources to endow peroxidase-like activity for Ti₃C₂-MXene, but also can solve the problems of high cost, long cycle and high operation difficulty of traditional uric acid detection methods and has the advantages of convenience, rapidness, accuracy, high efficiency and low cost. The detection range of uric acid is 5-400 μM. The method can realize accurate quantitative detection in actual samples, and has wide application prospects.

To better understand the present invention, the present invention is further described in detail below by the following embodiments, but shall not be interpreted as a limitation to the present invention. Some non-essential improvements and adjustments made by those skilled in the art according to the contents of the present invention shall also be deemed to fall within the protection scope of the present invention,

The technical solution of the present invention is further described below in combination with specific embodiments.

### Embodiment 1

A method for detecting uric acid through simulation of peroxidase activity based on, a nitrogen-sulfur co-doped Ti₃C₂-MXene nanosheet comprises the following steps:

### 1) Preparation of Ti₃C₂-MXene Nanosheet

slowly adding 5 g of MAX phase ceramic powder into 50 mL of hydrofluoric acid with mass fraction of 40%, and carrying out a reaction for 24 h at room temperature by magnetic stirring at a stirring speed of 800 r/min; after the reaction, washing and centrifuging a corrosion product till pH of supernate is greater than 6; washing with absolute ethanol for 5 times; and finally, placing the product in a vacuum oven of 60° C. for 12 h to obtain a Ti₃C₂-MXene nanosheet.

### 2) Preparation of Nitrogen-Sulfur Co-Doped Ti₃C₂-MXene Nanosheet

grinding and evenly mixing the Ti₃C₂-MXene nanosheet obtained in step 1) and thiourea according to a mass ratio of 1: 3; then, heating the mixture in an Ar gas atmosphere furnace to 500° C.; after heat preservation for 4 h, cooling in the furnace to room temperature; grinding the product again, and centrifuging with deionized water till the pH value of the supernate is close to 7; and finally drying the product to obtain the nitrogen-sulfur co-doped Ti₃C₂-MXene nanosheet.

### 3) Peroxidase-like Activity of Nitrogen-Sulfur Co-Doped Ti₃C₂-MXene Nanosheet

successively adding 100 μL of TMB solution (20 mM), 100 μL of hydrogen peroxide solution (50 mM) and 100 μL of nitrogen-sulfur co-doped Ti₃C₂-MXene nanosheet (0.2 mg/mL) into 1700 μL of disodium hydrogen phosphate-citrate buffer; evenly mixing a reaction system; after reaction for 10 min, determining the UV-VIS absorption spectrum of the mixed solution with an ultraviolet-visible spectrophotometer; and recording an absorbance value at a wavelength of 652 nm.

### 4) Colorimetric Detection for Uric Acid

successively adding 100 μL of nitrogen-sulfur co-doped Ti₃C₂-MXene nanosheet (0.2 mg/mL), 100 μL of uric acid solution (100 μM), 50 μL of hydrogen peroxide solution (50 mM) and 100 μL of TMB (20 mM) into 1600 μL of disodium hydrogen phosphate-citrate buffer; evenly mixing a reaction system and incubating in a water bath of 40° C. for 10 min; determining the UV-VIS absorption spectrum of the mixed solution with an ultraviolet-visible spectrophotometer; and recording the absorbance at a wavelength of 652 nm.

### Embodiment 2

A method for detecting uric acid through simulation of peroxidase activity based on a nitrogen-sulfur co-doped Ti₃C₂-MXene nanosheet comprises the following steps:

### 1) Preparation of Ti₃C₂-MXene Nanosheet

slowly adding 8g of MAX phase ceramic powder into 80mL of hydrofluoric acid with mass fraction of 50%, and carrying out a reaction for 12h at room temperature by magnetic stirring at a stirring speed of 1000 r/min; after the reaction, washing and centrifuging a corrosion product till pH of supernate is greater than 6; washing with absolute ethanol for 5 times; and finally, placing the product in a vacuum oven of 80° C. for 15h to obtain a Ti₃C₂-MXene nanosheet.

### 2) Preparation of Nitrogen-Sulfur Co-doped Ti₃C₂-MXene Nanosheet

grinding and evenly mixing the Ti₃C₂-MXene nanosheet obtained in step 1) and thiourea according to a mass ratio of 1: 2 then heating the mixture in an Ar gas atmosphere furnace to 600° C.; after heat preservation for 5 h, cooling in the furnace to room temperature; grinding the product again, and centrifuging with deionized water till the pH value of the supernate is close to 7; and finally drying the product to obtain the nitrogen-sulfur co-doped Ti₃C₂-MXene nanosheet

### 3) Peroxidase-like Activity of Nitrogen-Sulfur Co-doped Ti₃C₂-MXene Nanosheet

successively adding 50 μL of TMB solution (10 mM), 50 μL of hydrogen peroxide solution (50 mM) and 100 μL of nitrogen-sulfur co-doped Ti₃C₂-MXene nanosheet (0.5 mg/mL) into 1850 μL, of disodium hydrogen phosphate-citrate buffer; evenly mixing a reaction system; after reaction for 15 min, determining the UV-VIS absorption spectrum of the mixed solution with an ultraviolet-visible spectrophotometer; and recording an absorbance value at a wavelength of 652 nm.

### 4) Colorimetric Detection for Uric Acid

successively adding 50 μL, of nitrogen-sulfur co-doped Ti₃C₂-MXene nanosheet (0.5 mg/mL), 50 μL, of uric acid solution (200 μM), 50 μL of hydrogen peroxide solution (40 mM) and 50 μL of TMB (10mM) into 1800 μL of disodium hydrogen phosphate-citrate buffer; evenly mixing a reaction system and incubating in a water bath of 50° C. for 15 min; determining the UV-VIS absorption spectrum of the mixed solution with an ultraviolet-visible spectrophotometer; and recording the absorbance at a wavelength of 652 nm.

### Embodiment 3

A method for detecting uric acid through simulation of peroxidase activity based on a nitrogen-sulfur co-doped Ti₃C₂-MXene nanosheet comprises the following steps:

### 1) Preparation of Ti₃C₂-MXene Nanosheet

slowly adding 6 g of MAX phase ceramic powder into 70 mL of hydrofluoric acid with mass fraction of 50%, and carrying out a reaction for 12 h at room temperature by magnetic stirring at a stirring speed of 1000 r/min; after the reaction, washing and centrifuging a corrosion product till pH of supernate is greater than 6; washing with absolute ethanol for 7 times; and finally, placing the product in a vacuum oven of 80° C. for 15 h to, obtain a Ti₃C₂-MXene nanosheet

### 2) Preparation of Nitrogen-Sulfur Co-doped Ti₃C₂-MXene Nanosheet

grinding and evenly mixing the Ti₃C₂-MXene nanosheet obtained in step 1) and thiourea according to a mass ratio of 1: 4; then heating the mixture in an Ar gas atmosphere furnace to 550° C.; after heat preservation for 8 h, cooling in the furnace to room temperature; grinding the product again, and centrifuging with deionized water till the pH value of the supernate is close to 7; and finally drying the product to obtain the nitrogen-sulfur co-doped Ti₃C₂-MXene nanosheet.

### 3) Peroxidase-like Activity of Nitrogen-Sulfur Co-doped Ti₃C₂-MXene Nanosheet

successively adding 50 of TMB solution (10 mM), 50 μL, of hydrogen peroxide solution (50 mM) and 100 μL of nitrogen-sulfur co-doped Ti₃C₂-MXene nanosheet (0.4 mg/mL) into 1850 μL of disodium hydrogen phosphate-citrate buffer; evenly mixing a reaction system; after reaction for 20 min. determining the UV-VIS absorption spectrum of the mixed solution with an ultraviolet-visible spectrophotometer; and recording an absorbance value at a wavelength of 652 nm.

### 4) Colorimetric Detection for Uric Acid

successively adding 50 μL of nitrogen-sulfur co-doped Ti₃C₂-MXene nanosheet (0.5 mg/mL), 50 μL of uric acid solution (200 μM), 50 of hydrogen peroxide solution (40 mM) and 50 μL of TMB (10 mM) into 1800 μL of disodium hydrogen phosphate-citrate buffer; evenly mixing a reaction system and incubating in a water bath of 50° C. for 15 min; determining the UV-VIS absorption spectrum of the mixed solution with an ultraviolet-visible spectrophotometer; and recording the absorbance at a wavelength of 652 nm.

### Embodiment 4

A method for detecting uric acid through simulation of peroxidase activity based on a nitrogen-sulfur co-doped Ti₃C₂-MXene nanosheet comprises the following steps:

### 1) Preparation of Ti₃C₂-MXene Nanosheet

slowly adding 4g of MAX phase ceramic powder into 50 mL of hydrofluoric acid with mass fraction of 50%, and carrying out a reaction for 8h at room temperature by magnetic stirring at a stirring speed of 1000 r/min; after the reaction, washing and centrifuging a corrosion product till pH of supernate is greater than 6; washing with absolute ethanol for 7 times; and finally, placing the product in a vacuum oven of 60° C. for 10 h to obtain a Ti₃C₂-MXene nanosheet.

### 2) Preparation of Nitrogen-Sulfur Co-doped Ti₃C₂-MXene Nanosheet

grinding and evenly mixing the Ti₃C₂-MXene nanosheet obtained in step 1) and thiourea according to a mass ratio of 1: 4; then heating the mixture in an Ar gas atmosphere furnace to 600° C.; after heat preservation for 8 h, cooling in the furnace to room temperature; grinding the product again, and centrifuging with deionized water till the pH value of the supernate is close to 7; and finally drying the product to obtain the nitrogen-sulfur co-doped Ti₃C₂-MXene nanosheet.

### 3) Peroxidase-like Activity of Nitrogen-Sulfur Co-doped Ti₃C₂-MXene Nanosheet

successively adding 50 μL of TMB solution (10 mM), 50 μL of hydrogen peroxide solution (50 mM) and 100 μL of nitrogen-sulfur co-doped Ti₃C₂-MXene nanosheet (0.3 mg/mL) into 1850 μL of disodium hydrogen phosphate-citrate buffer; evenly mixing a reaction system; after reaction for 10 min, determining the UV-VIS absorption spectrum of the mixed solution with an ultraviolet-visible spectrophotometer; and recording an absorbance value at a wavelength of 652 nm.

### 4) Colorimetric Detection for Uric Acid

successively adding 50 μL of nitrogen-sulfur co-doped Ti₃C₂-MXene nanosheet (0.5 mg/mL), 50 μL of uric acid solution (200 μM), 50 μL of hydrogen peroxide solution (40 mM) and 50 μL of TMB (10 mM) into 1800 μL of disodium hydrogen phosphate-citrate buffer; evenly mixing a reaction system and incubating in a water bath of 50° C. for 15 min; determining the UV-VIS absorption spectrum of the mixed solution with an ultraviolet-visible spectrophotometer; and recording the absorbance at a wavelength of 652 nm.

The contents of the present invention are not limited to the contents of the above embodiments, and a combination of one or more embodiments can also achieve the purposes of the present invention.

In order to further verify the excellent effects of the present invention, the inventors also conduct the following experiment.

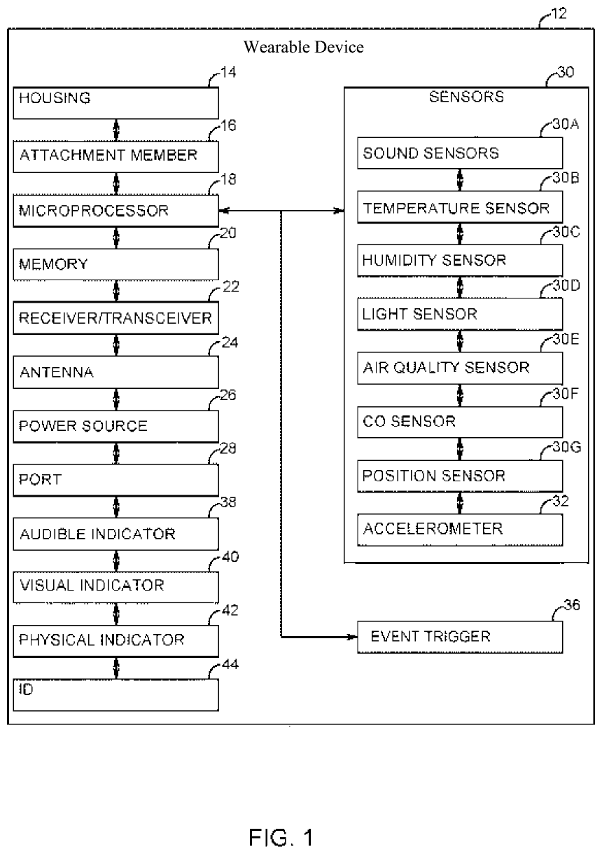

FIG. 1 shows an SEM diagram of a Ti₃C₂-MXene nanosheet prepared in embodiment 1. It can be seen that the Ti₃C₂-MXene nanosheet has an organ-shaped layered structure with a thickness of about 8 μm, and layer spacings are evenly distributed.

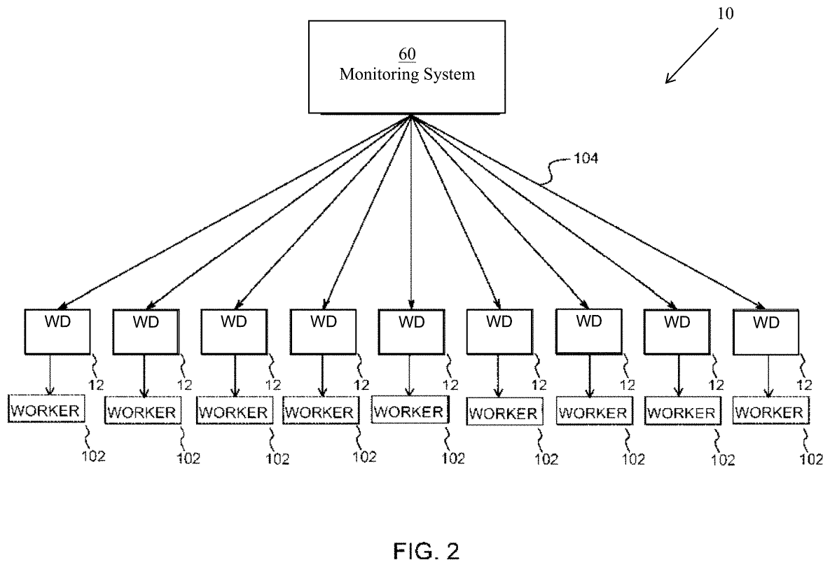

FIG. 2 shows XPS spectrograms of a nitrogen-sulfur co-doped Ti₃C₂-MXene nanosheet prepared in embodiment 1, wherein (a) XPS full spectrum indicates successful doping of elements N and S; (b) C is fine spectrum indicates the covalent bond structure of elements N and S and element C; (c) N is fine spectrum indicates that element N has the forms of pyrrole nitrogen, pyridine nitrogen, graphite nitrogen and nitrogen oxide; and (d) S 2p fine spectrum indicates the formation, of C-S-C covalent bond. It can be seen from FIG. 2 that elements Ti 2p, C1s, O1s, N1s and S2p exist in the nitrogen-sulfur co-doped Ti₃C₂-MXene.

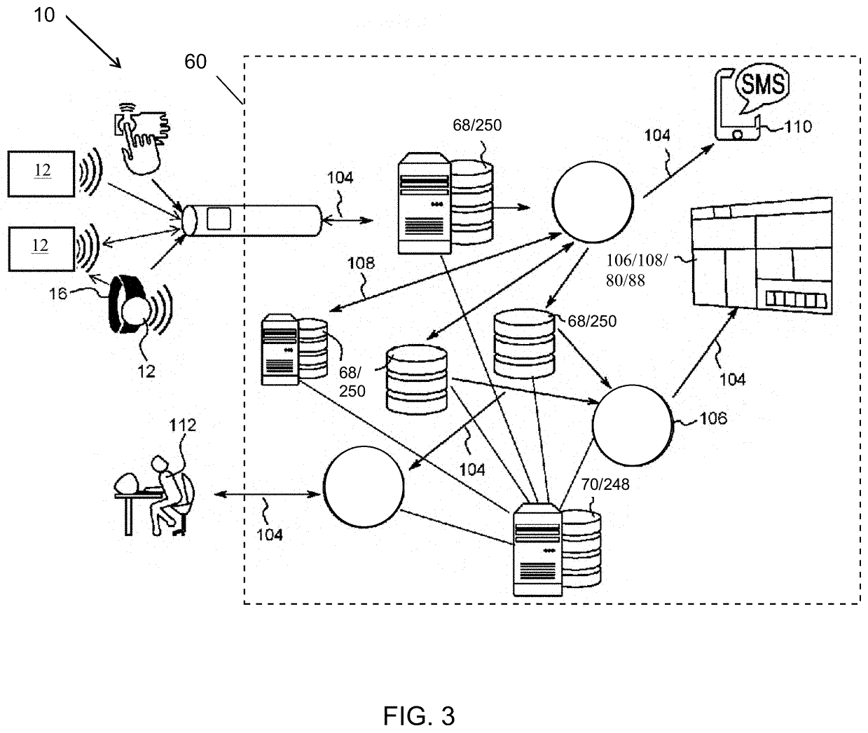

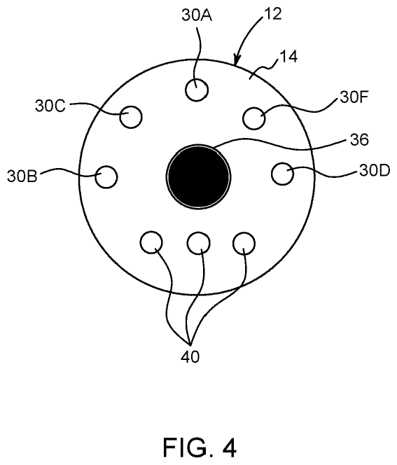

As shown in FIG. 3, the illustration represents the color change of solutions in different reaction systems after 10 min. Illustration a is TMB+H₂O₂+NS-Ti₃C₂ system, illustration b is TMB +NS-Ti₃C₂ system, illustration c is TMB+H₂O₂ system, and illustration d is TMB system. It can be seen from FIG. 3 that when H₂O₂ exists, the nitrogen-sulfur co-doped Ti₃C₂-MXene nanosheet can catalyze H₂O₂ to oxidize colorless TMB into a blue product, and the maximum absorption peak is located at 652 nm. It indicates that the nitrogen-sulfur co-doped Ti₃C₂-MXene nanosheet of the present invention has peroxidase-like activity.

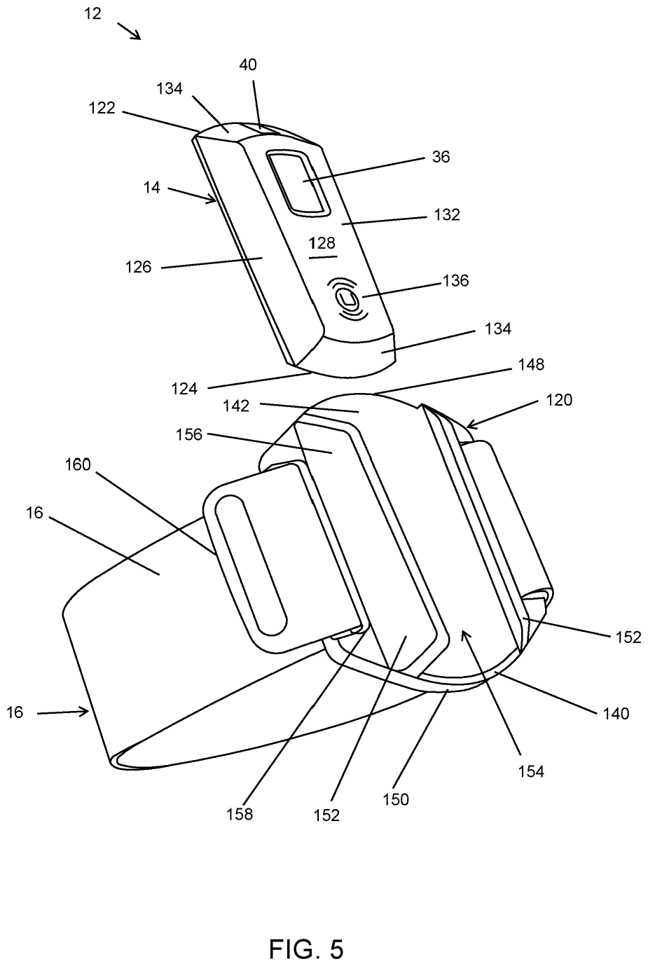

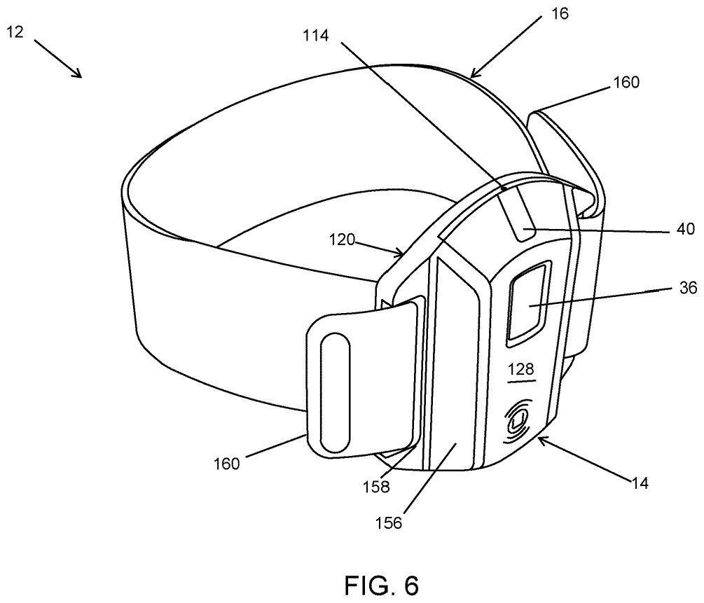

FIG. 4 and FIG. 5 show the steady-state kinetic experiments of the nitrogen-sulfur co-doped Ti₃C₂-MXene nanosheet with TMB and H₂O₂ as substrates in embodiment 1. According to Lineweaver-Burk diagram (illustration), Km values of the nitrogen-sulfur co-doped Ti₃C₂-MXene nanosheet, with TMB and H₂O₂ as substrates can be calculated as 0.132 mM and 5.46 mM respectively, indicating that the nitrogen-sulfur co-doped Ti₃C₂-MXene nanosheet has strong affinity for the substrates.

FIG. 6 shows the absorbance of the nitrogen-sulfur co-doped Ti₃C₂-MXene nanosheet in embodiment 2 after incubation with uric acid of different concentrations. It can be seen from the figure that the absorbance at 652 nm is gradually decreased with the increase of uric acid concentrations. In the range of 5-400 μM, there is a good linear correlation between a colorimetric signal and the uric acid concentration, and a detection limit of uric acid is 1 μM according to a 3-time signal-to-noise ratio method. These results indicate that a uric acid colorimetric sensor based on the nitrogen-sulfur co-doped Ti₃C₂-MXene nanosheet has a wide detection range and a low detection limit.

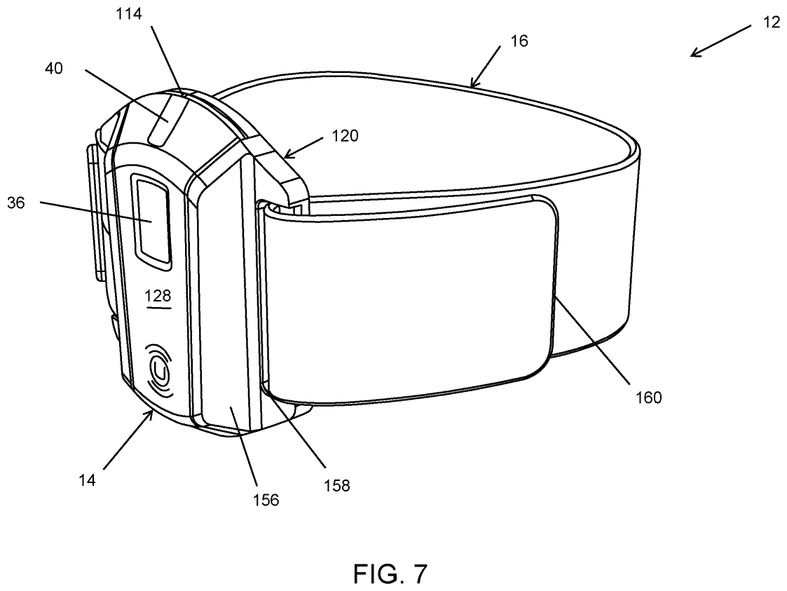

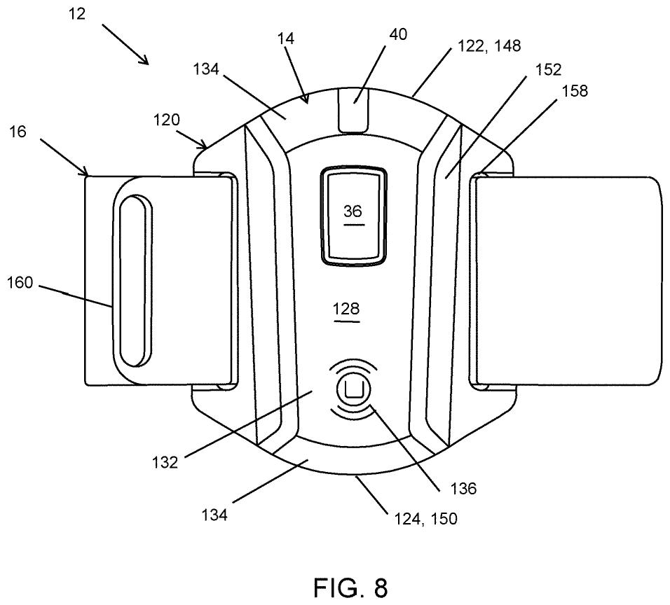

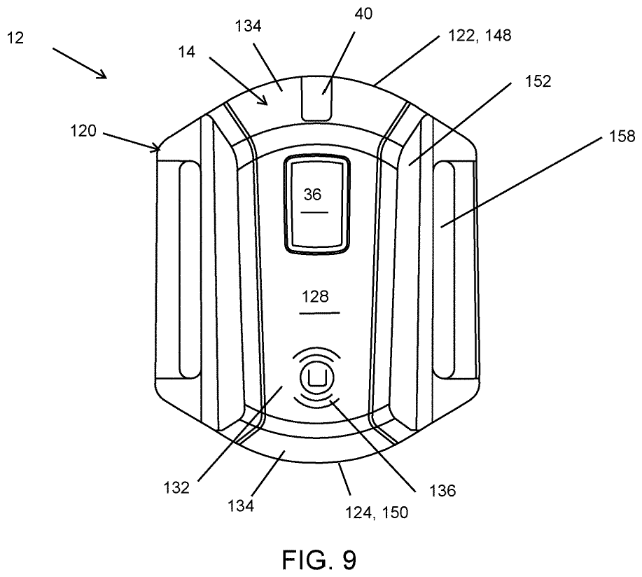

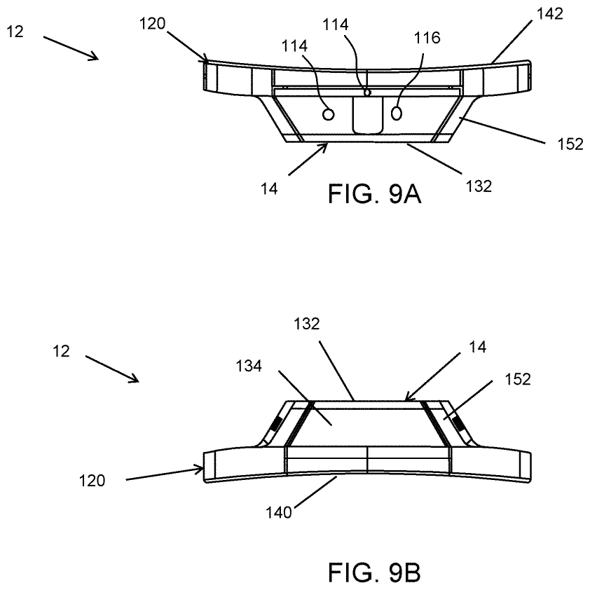

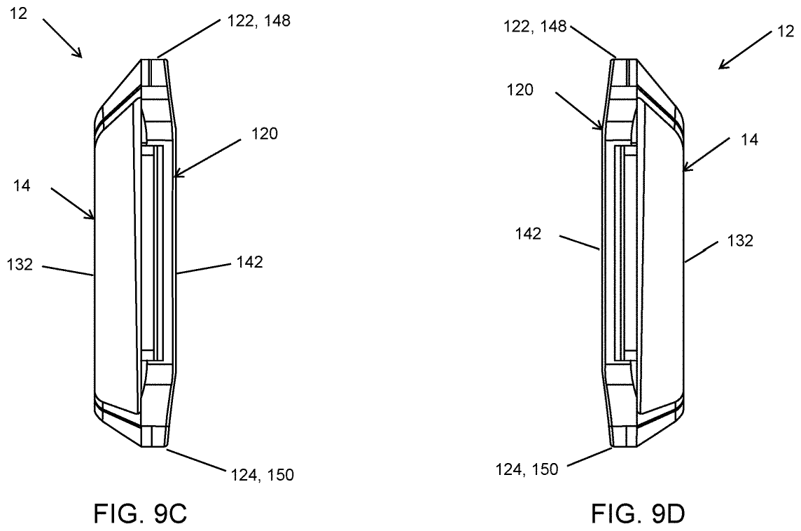

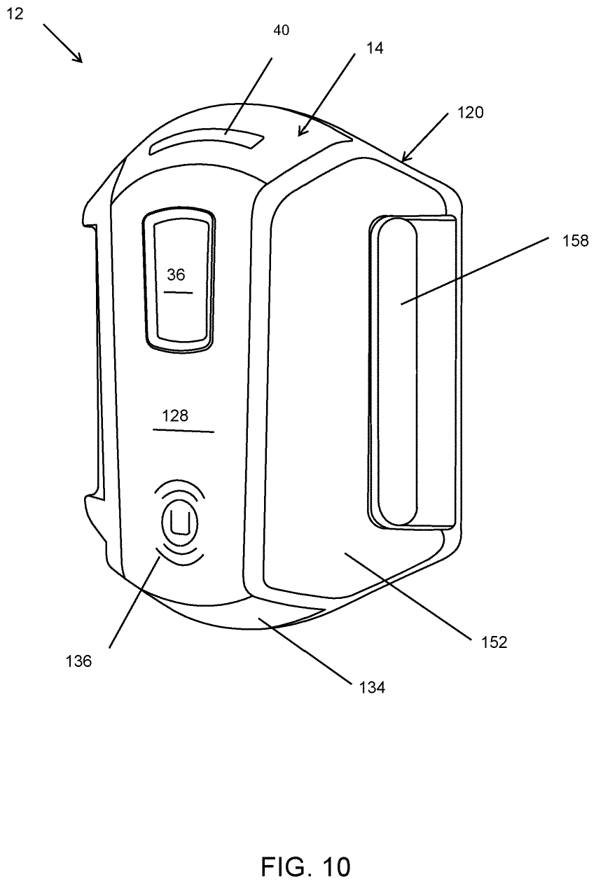

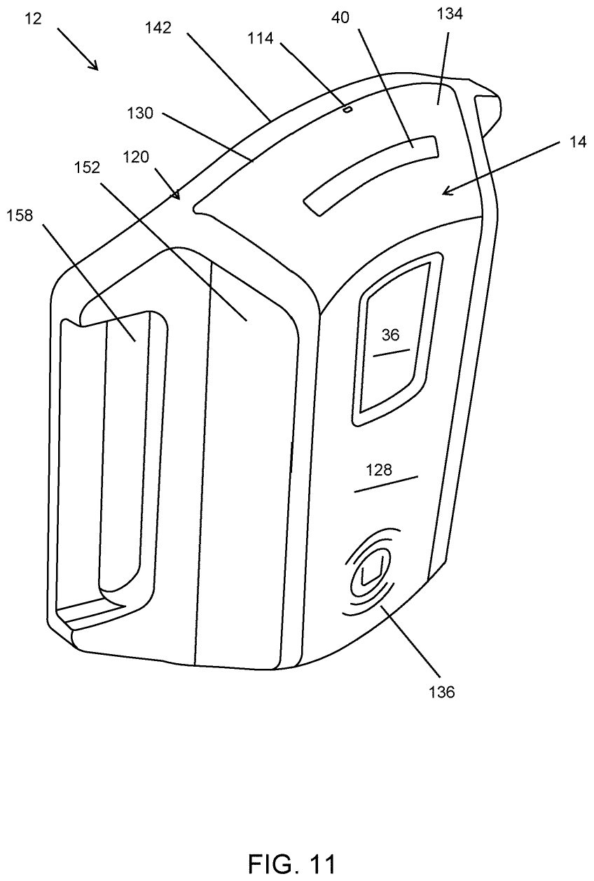

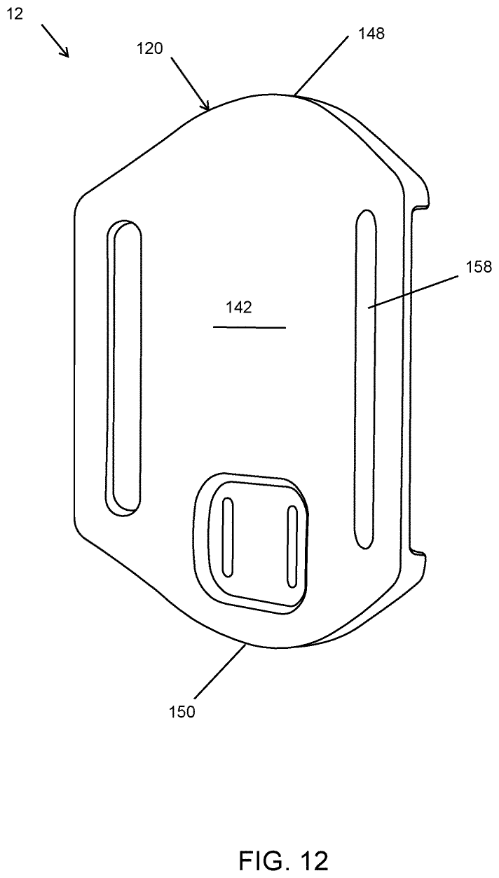

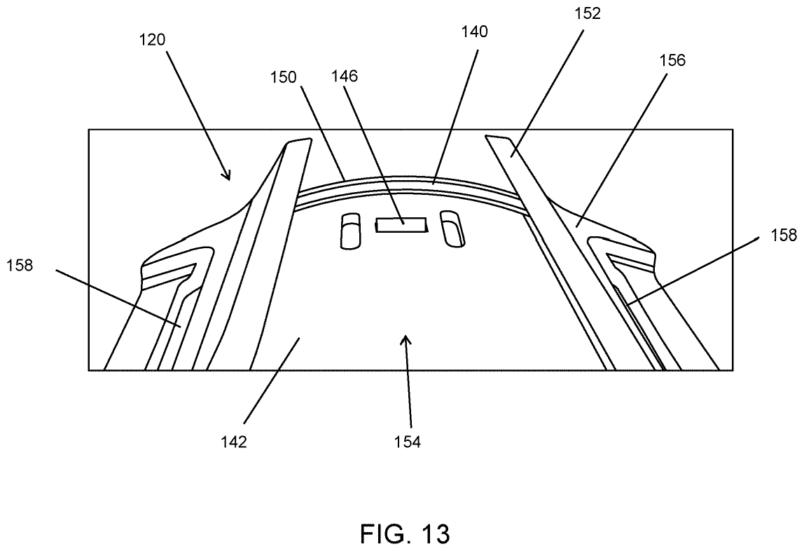

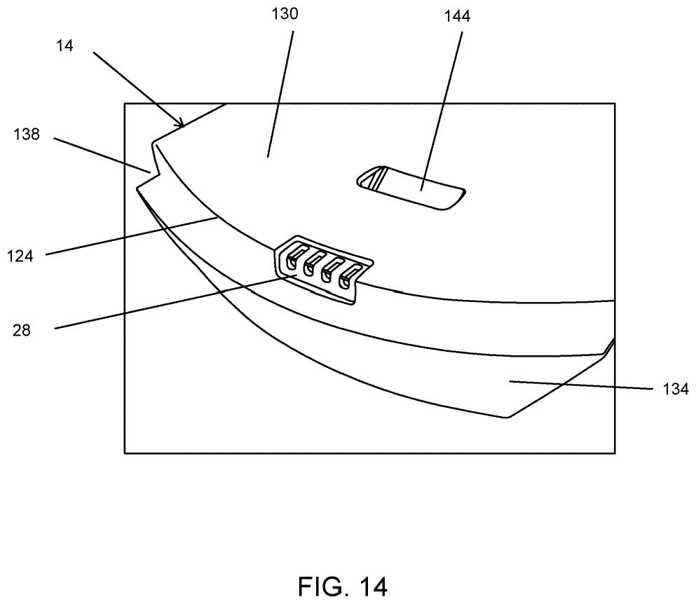

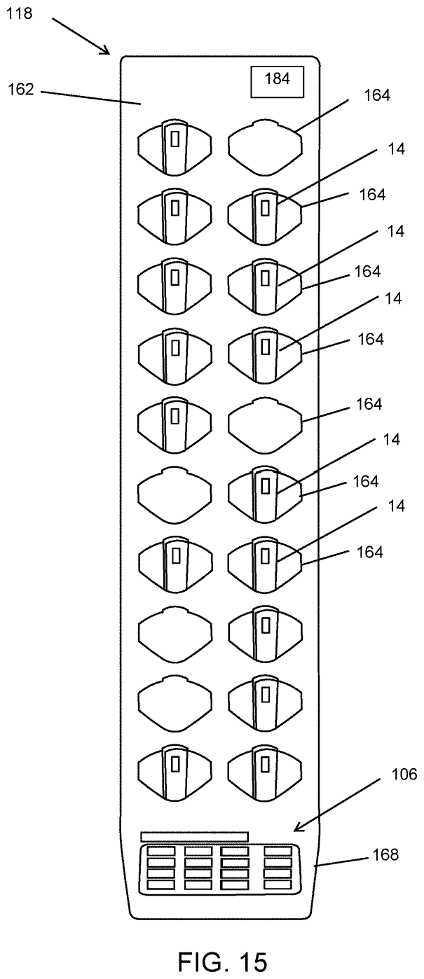

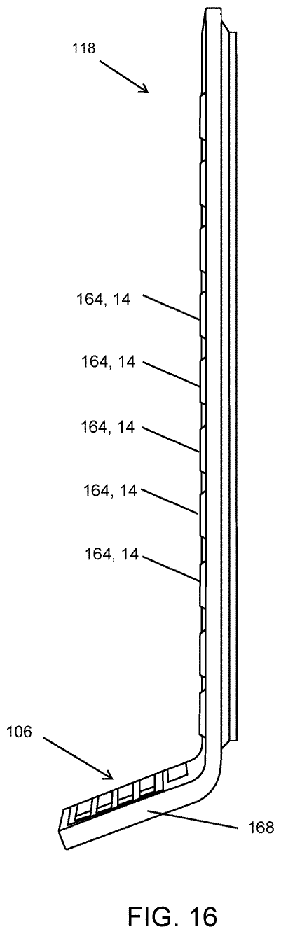

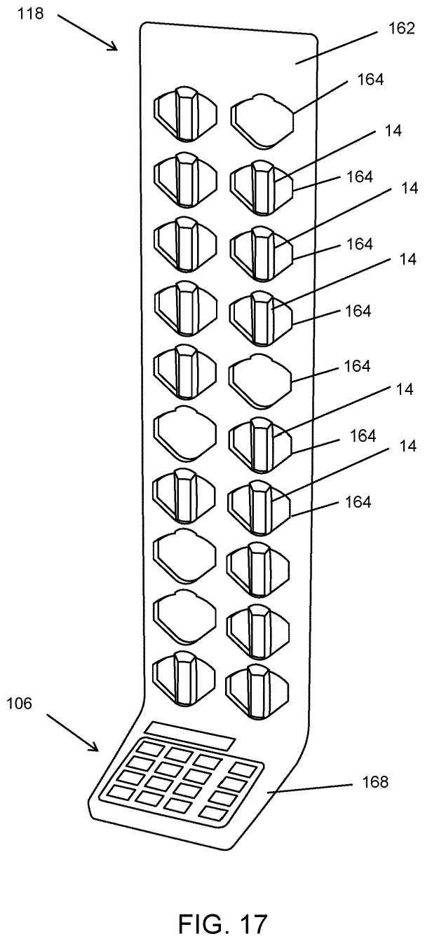

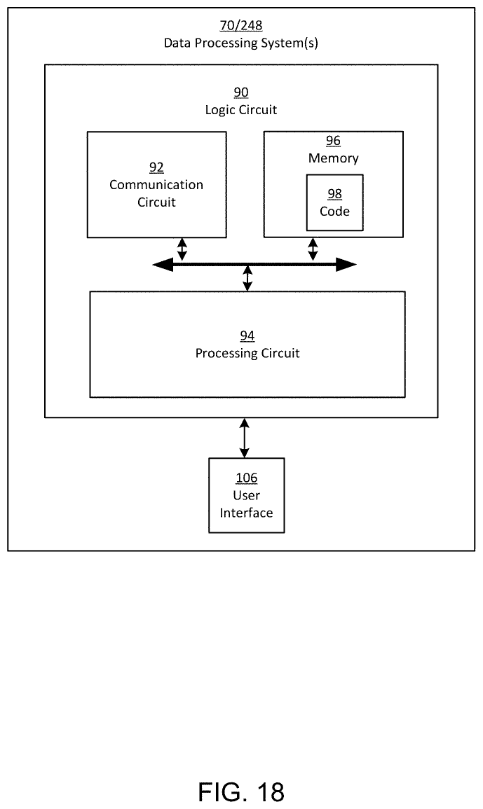

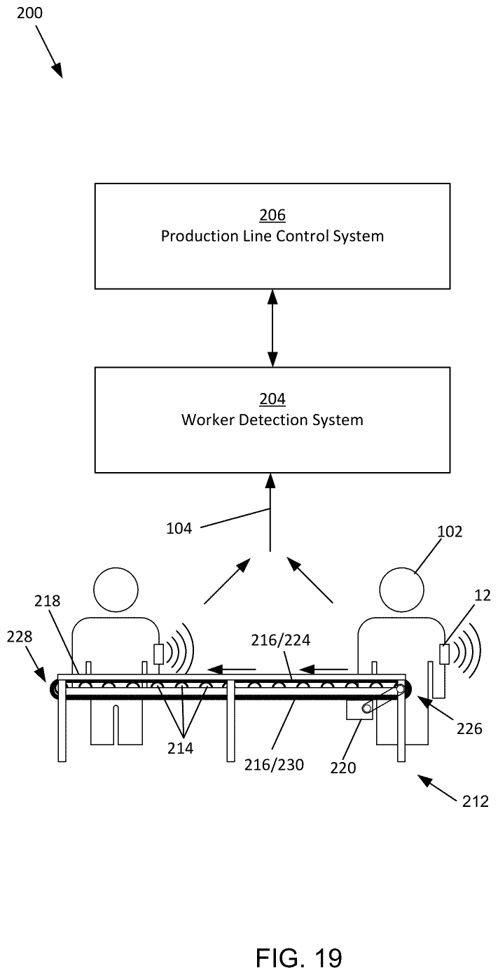

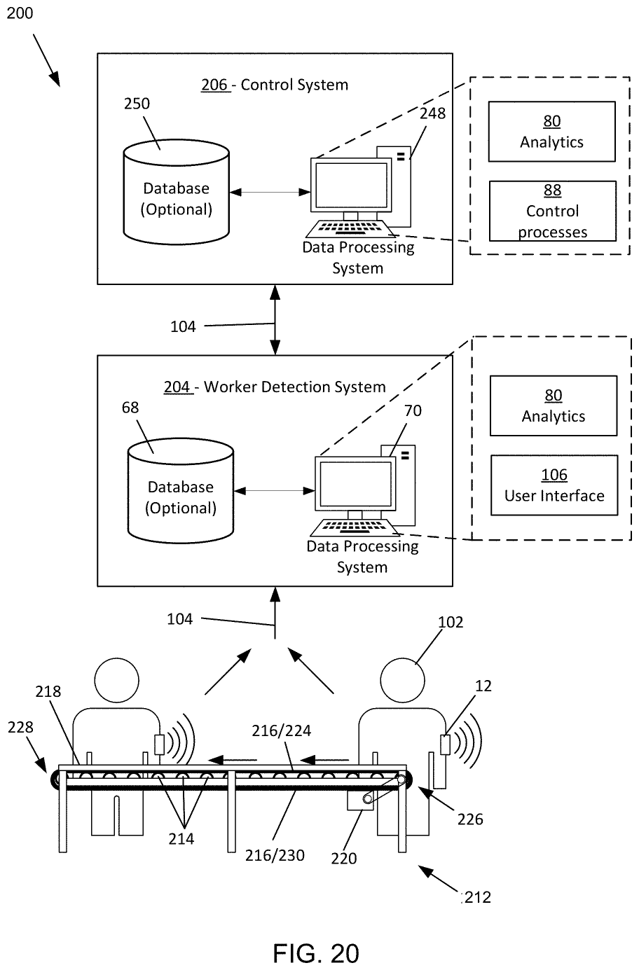

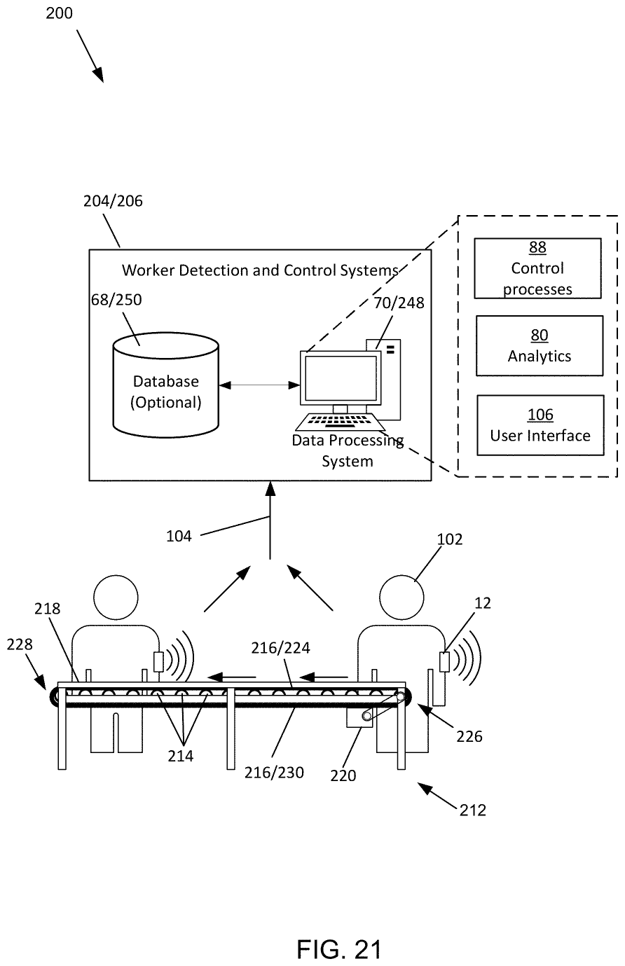

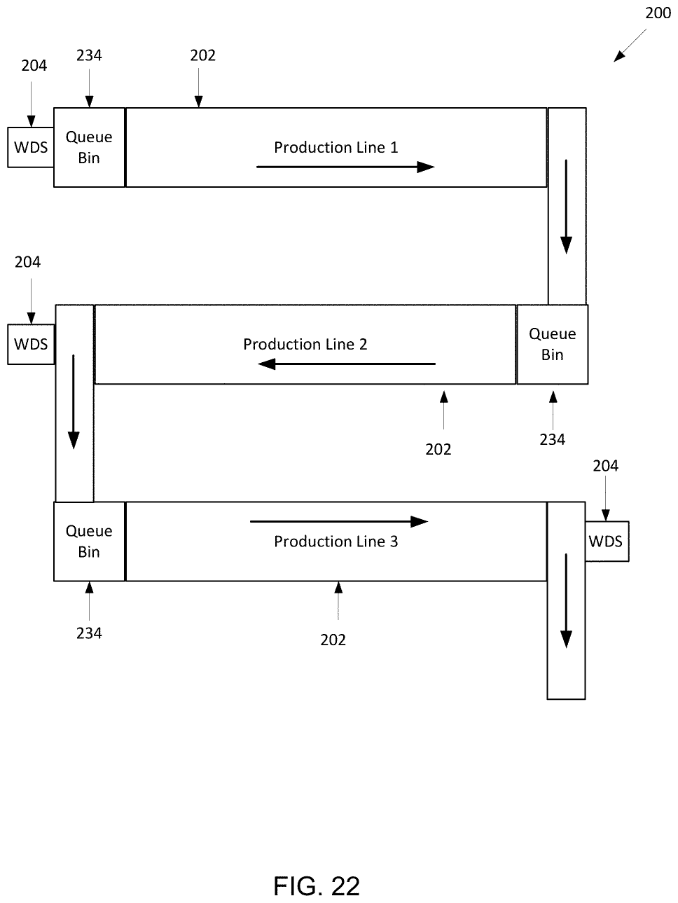

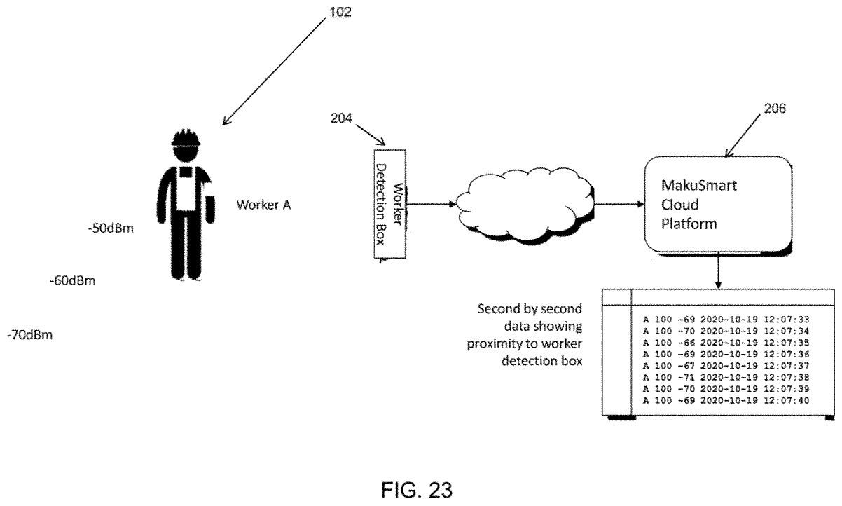

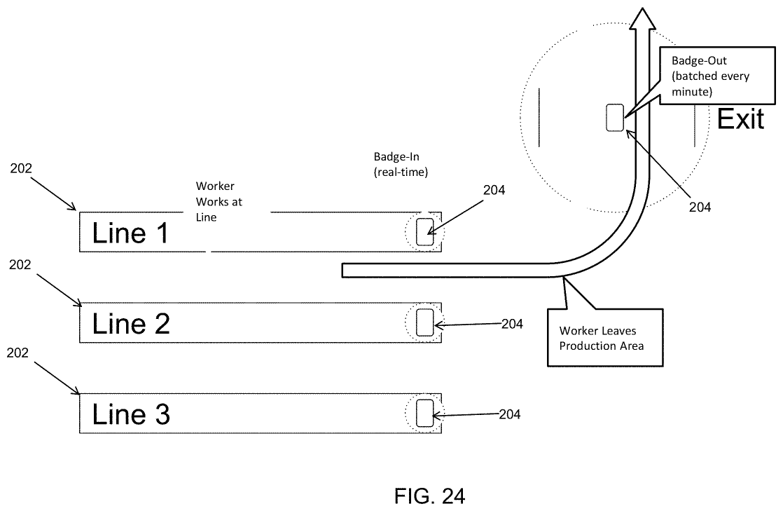

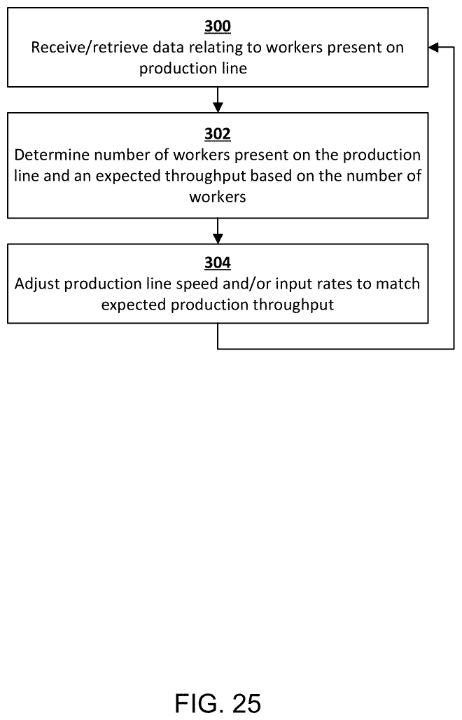

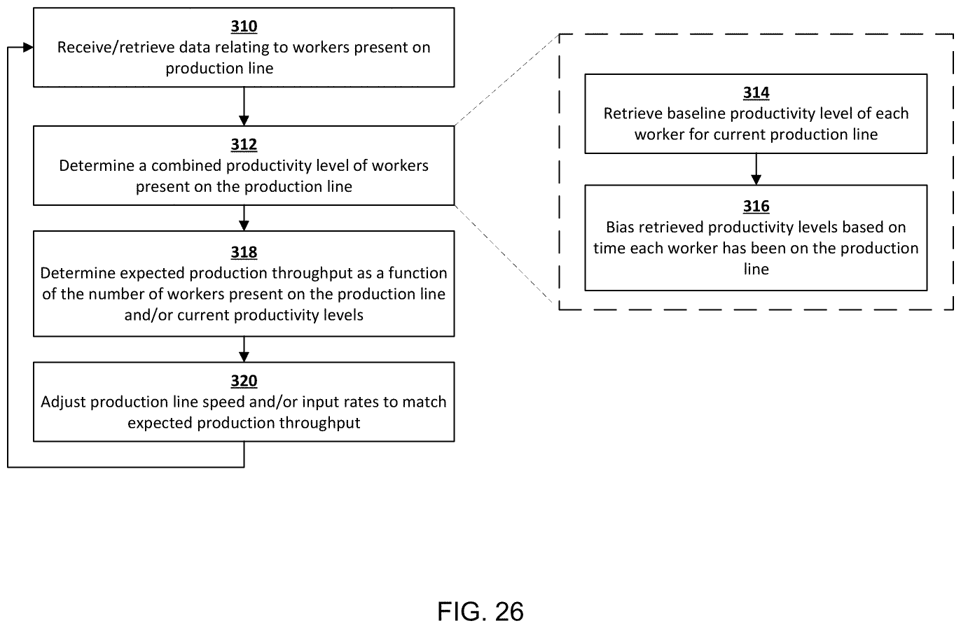

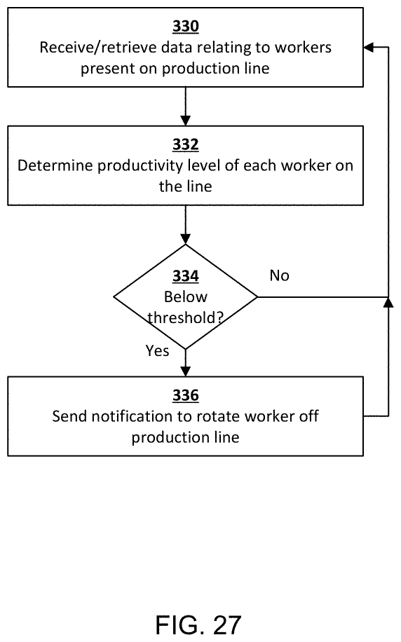

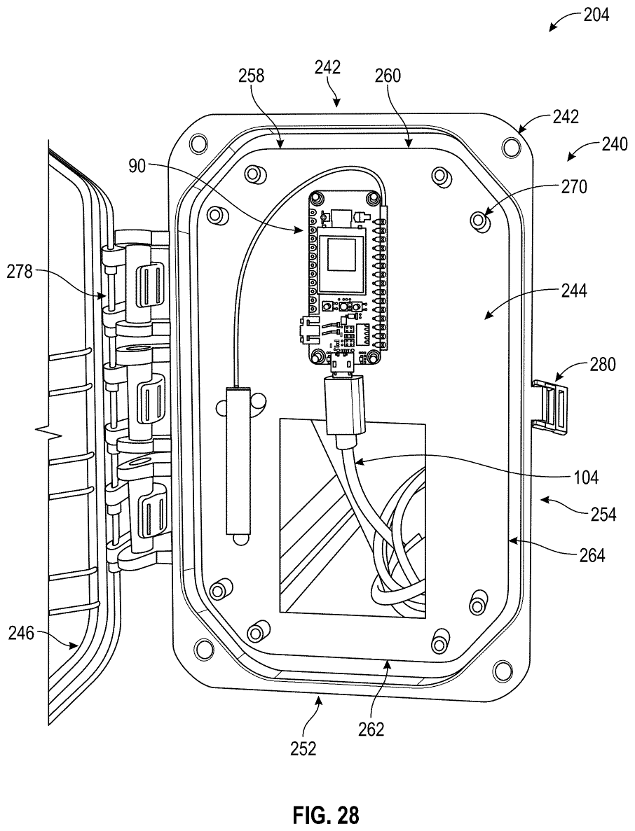

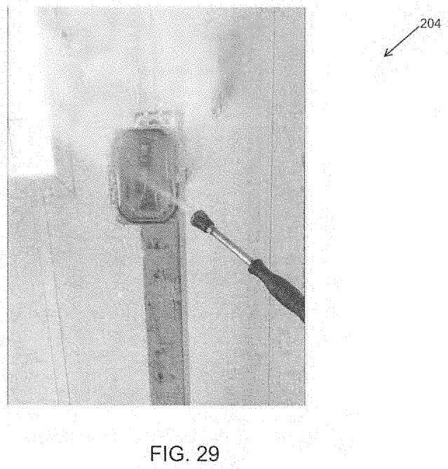

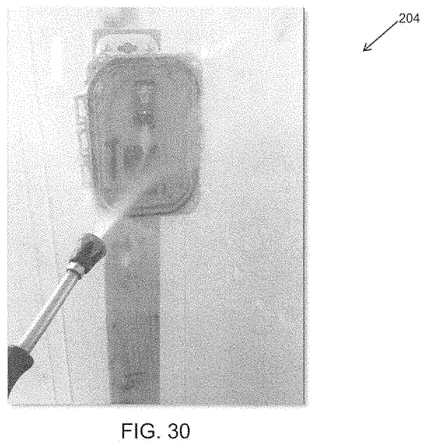

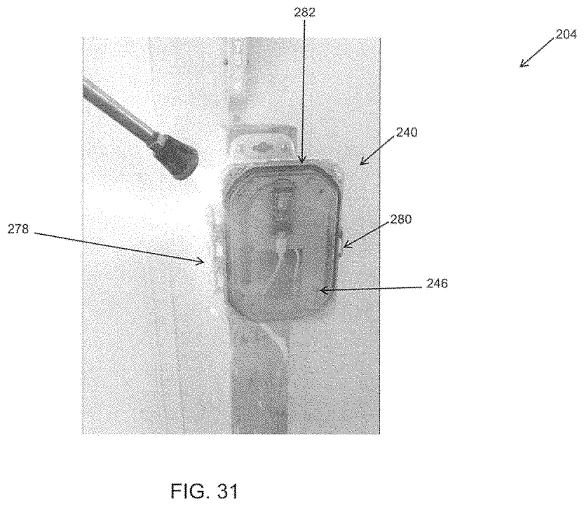

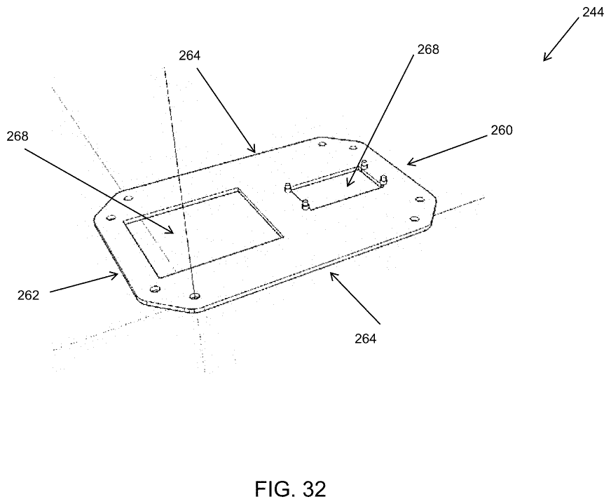

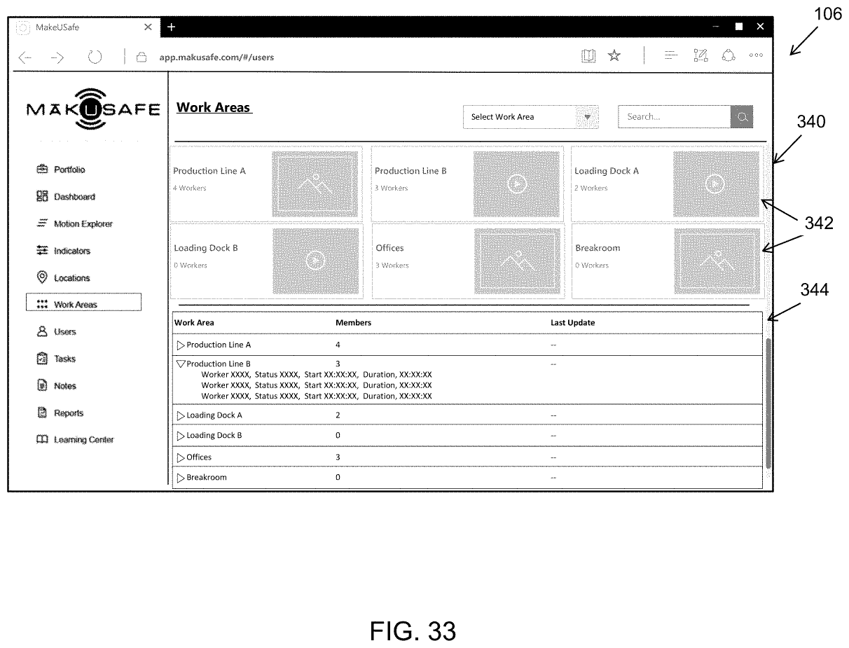

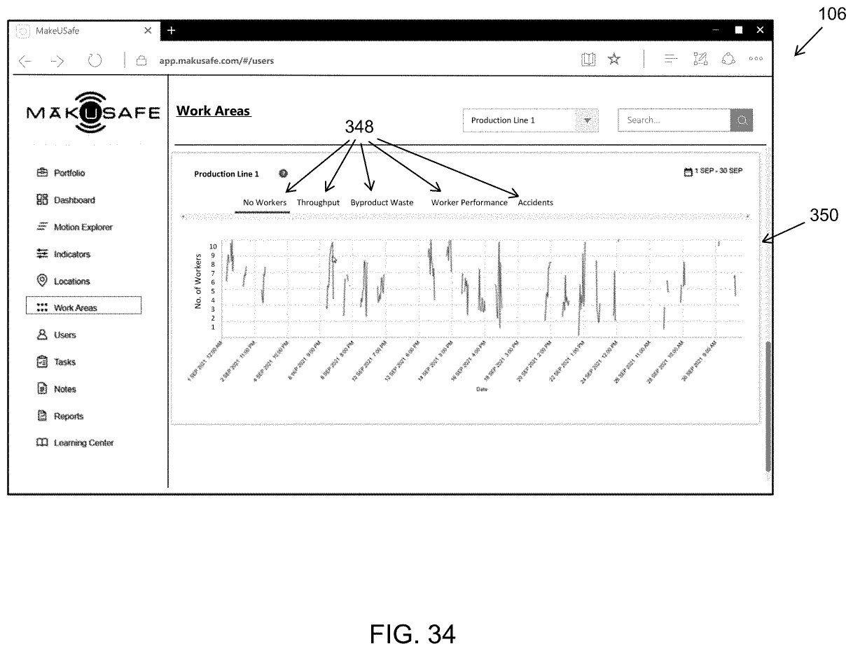

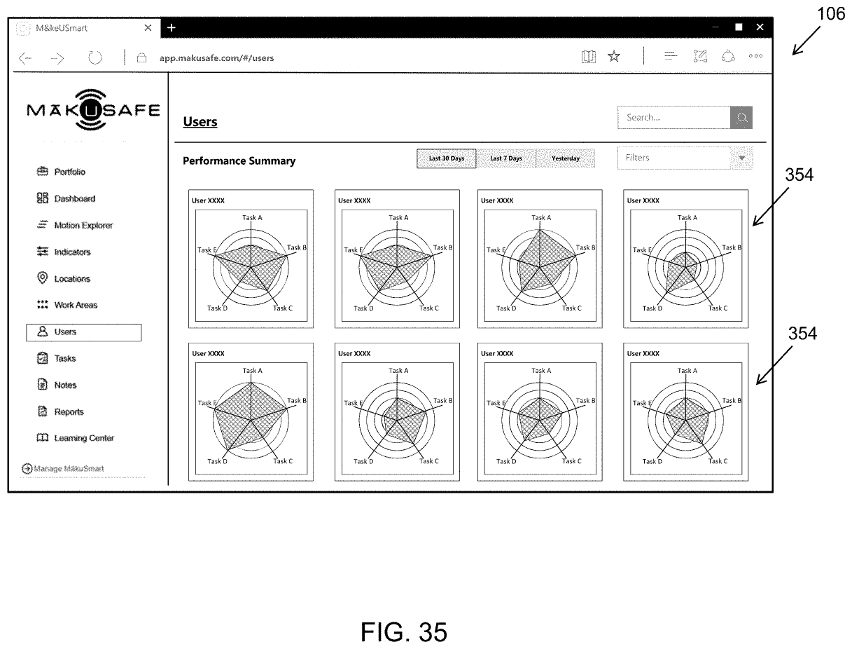

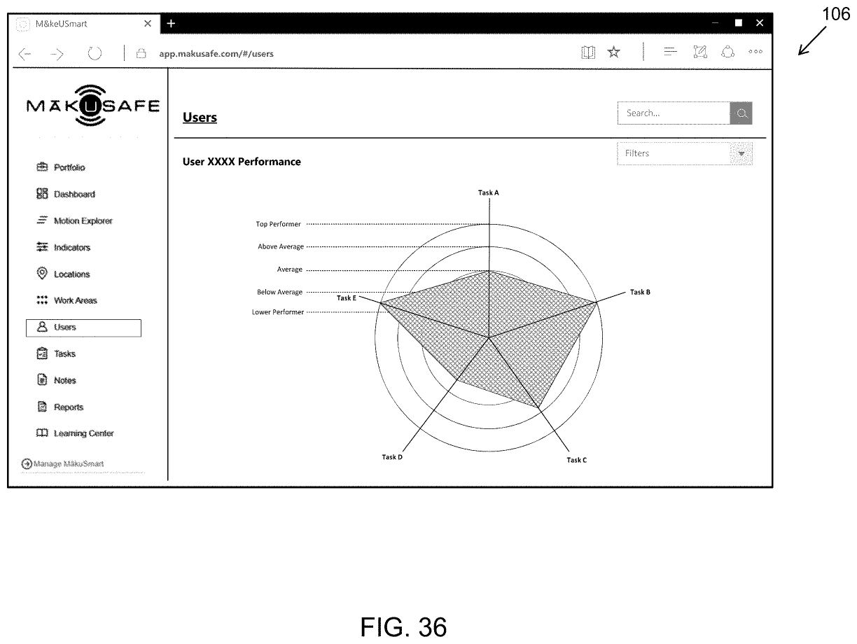

FIG. 7 shows the selectivity of the nitrogen-sulfur co-doped Ti₃C₂-MXene nanosheet for uric acid detection in embodiment 2. Δ_{A}=-A₁−A₂. A₁ represents the absorbance value of the reaction system after adding TMB, and A₂ represents the absorbance value of the reaction system before adding TMB. The illustrations indicate the influence of different interferents on the color change of the solutions in the reaction system. As shown in FIG. 7, the sensing platform shows excellent selectivity for uric acid.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A nitrogen-sulfur co-doped Ti₃C₂-MXene nanosheet, wherein the nitrogen-sulfur co-doped Ti₃C₂-MXene nanosheet is prepared by a one-step method with thiourea as a source of heteroatom doping; the nitrogen-sulfur co-doped Ti₃C₂-MXene nanosheet has an organ-shaped layered structure with a thickness of 6-10 μm, and doped elements are evenly distributed on the nanosheet

2. The nitrogen-sulfur co-doped Ti₃C₂-MXene nanosheet according to claim 1, wherein the nitrogen-sulfur co-doped Ti₃C₂-MXene nanosheet has peroxidase-like activity.

3. A preparation method of the nitrogen-sulfur co-doped Ti₃C₂-MXene nanosheet of claim 1, the method specifically comprising the following steps:
S1 slowly adding MAX phase ceramic powder to hydrofluoric acid, and stirring by magnetic force at room temperature to react after the reaction, washing and centrifuging a corrosion product, washing with absolute ethanol for 3-8 times; and finally, drying the product in a vacuum oven to obtain a Ti₃C₂-MXene nanosheet;
S2 grinding and evenly mixing the Ti₃C₂-MXene nanosheet obtained in step 1) and thiourea; then roasting the mixture in an Ar gas atmosphere furnace, and then cooling in the furnace to room temperature; grinding the product again, and centrifugally washing the product with deionized water; and finally drying the product to obtain the nitrogen-sulfur co-doped Ti₃C₂-MXene nanosheet

4. The preparation method of the nitrogen-sulfur co-doped Ti₃C₂-MXene nanosheet according to claim 3, wherein in step 1), the reaction mass ratio of the MAX phase ceramic powder and the hydrofluoric acid is 1:4 to 1:8, stirring reaction time is 8-24 h, and a stirring rate is 500-1000 r/min.

5. The preparation method of the nitrogen-sulfur co-doped Ti₃C₂-MXene nanosheet according to claim 3, wherein drying temperature in the vacuum oven is 40° C.-80° C., and drying time is 8-16 h.

6. The preparation method of the nitrogen-sulfur co-doped Ti₃C₂-MXene nanosheet according to claim 3, wherein in step 2), the mixing mass ratio of the Ti₃C₂-MXene nanosheet and the thiourea is (¼-½): 1, roasting temperature is 300° C.-700° C., and temperature retention time is 4-8 h.

7. An application of the nitrogen-sulfur co-doped Ti₃C₂-MXene nanosheet of claim 1 in a method for detecting uric acid through simulation of peroxidase activity.

8. The application according to claim 7, wherein the nitrogen-sulfur co-doped Ti₃C₂-MXene nanosheet has peroxidase-like activity, and the method for detecting uric acid is a colorimetric detection method.

9. The application according to claim 8, wherein the colorimetric detection method for uric acid comprises the following steps:
successively adding a nitrogen-sulfur co-doped Ti₃C₂-MXene nanosheet, a uric acid solution, a hydrogen peroxide solution and TMB into disodium hydrogen phosphate-citrate buffer; evenly mixing a reaction system and incubating in a water bath; then determining the UV-VIS absorption spectrum of the mixed solution; and recording absorbance, at a wavelength of 652 nm.

10. The application according to claim 9, wherein after mixing, the incubation temperature of the reaction system is 30-50° C., and reaction time is 5-20 min; and the concentration of the uric acid solution in the reaction system is 5 μM, 10 μM, 80 μM, 100 μM, 150 μM, 250 μM, 300 μM, 350 μM or 400 μM.

11. An application of the nitrogen-sulfur co-doped Ti₃C₂-MXene nanosheet prepared by the method of claim 3 in a method for detecting uric acid through simulation of peroxidase activity.
